(19) United States
(12) Patent Application Publication
(10) Pub. No.: US 2026/0166221 A1
(43) Pub. Date: Jun. 18, 2026

(54) INSULIN PATCH PUMP SYSTEM WITH IMPROVED FLUIDIC CONNECTION

(71) Applicant: Tandem Diabetes Care, Inc., San Diego, CA (US)

(72) Inventor: Vincent Vaucher, St-Sulpice (CH)

(21) Appl. No.: 19/418,335

(22) Filed: Dec. 12, 2025

Related U.S. Application Data
(60) Provisional application No. 63/733,647, filed on Dec. 13, 2024.

(51) Int. Cl. A61M 5/162; A61M 5/142

(52) U.S. Cl. CPC A61M 5/162; A61M 5/14248; A61M 2005/14252

(57) ABSTRACT

Provided herein are systems and methods for delivering medication, such as insulin, that are user-friendly, environmentally-friendly, lower cost, discreet, less prone to errors, and/or that deliver precise, repeatable doses of medication, as well as accessories for applying and managing the same. In embodiments, the system includes a wearable insulin pump having a patch-style form factor for adhesion to a user's body surface.

### PRIORITY CLAIM

The present application claims the benefit of U.S. Provisional Patent Application No. 63/733,647 filed Dec. 13, 2024, which is hereby incorporated herein by reference in its entirety.

### TECHNICAL FIELD

The present disclosure relates generally to systems and methods for delivering medication such as insulin to a user, for example, wearable insulin pumps having a patch-style form factor for adhesion to a user's body surface.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

For patch pumps, a transcutaneous cannula generally needs to be inserted into the skin for delivering medication transcutaneously from an external fluid reservoir associated with the patch pump that is in fluid communication with the cannula. Thus, the cannula needs to be inserted into the skin before delivering the medication. Patch pump systems may include an applicator that can simultaneously insert the cannula into the user's skin and affix an adhesive pad onto the user's skin that holds the patch pump.

### SUMMARY

Provided herein are systems and methods for delivering medication, such as insulin, that are user-friendly, environmentally-friendly, lower cost, discreet, less prone to errors, and/or that deliver precise, repeatable doses of medication, as well as accessories for applying and managing the same. In embodiments, the system includes a wearable insulin pump having a patch-style form factor for adhesion to a user's body surface.

In accordance with one aspect, a medication infusion system is provided that includes that includes a patch pump configured to be removably attached to a pad skeleton worn on the user's skin that delivers medication (e.g., insulin) from a cartridge through a cannula secured in the pad skeleton and inserted into the user's skin. The system disclosed herein provides a more secure and reliable connection from the cartridge, through the cannula and into the user's skin. In particular, the system disclosed herein provides a larger tolerance on insertion of the cannula into the pad skeleton to provide a more reliable connection between the cannula and the pad skeleton while controlling a positioning of the cannula when the pump is attached to ensure a precise positioning of an outflow needle that puts the cartridge into fluid communication with the cannula.

In an embodiment, a medication infusion system includes a pad skeleton having a pad attachment. An applicator can be configured to attach the pad skeleton onto a body of a user and to attach a cannula including a septum to the pad attachment. The applicator can cause a variable positioning of the cannula with respect to the pad attachment on insertion of the cannula to provide a secure attachment between the cannula and the pad attachment. A patch pump can include a reservoir configured to contain a medicament and be configured to be attached to the pad skeleton to cause an outflow needle in fluid communication with the reservoir to pierce the septum to cause the reservoir to be in fluid communication with the cannula. The patch pump can be configured to control the variable positioning of the cannula as the septum is pierced by the outflow needle.

In an embodiment, a medication infusion system can include a pad skeleton having a pad attachment and an applicator configured to attach the pad skeleton onto a body of a user and to attach a cannula assembly to the pad attachment. The applicator can be configured to insert the cannula assembly into a skin of the user to an insertion depth such that a natural reaction of the skin following insertion causes the cannula assembly to move in a direction opposite of a direction of insertion to interlock the cannula assembly with the pad attachment.

In embodiments, a medication infusion system can include a pad skeleton having a pad attachment configured to receive a cannula assembly having a septum therein, the cannula assembly having a variable positioning with respect to the pad attachment. A patch pump including a reservoir configured to contain a medicament can be configured to be attached to the pad skeleton to cause an outflow needle in fluid communication with the reservoir to pierce the septum to cause the reservoir to be in fluid communication with the cannula assembly, wherein the patch pump is configured to control the variable positioning of the cannula assembly as the septum is pierced by the outflow needle.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A and 3B are, respectively, a perspective view and an exploded view of the pad and applicator of FIG. 1.

FIGS. 4A-4B depict an exemplary cannula for delivering medication transcutaneously.

FIGS. 5A-5D schematically depict a portion of an applicator of a medical infusion system.

FIGS. 6A-6C schematically depict a portion of an applicator of a medical infusion system.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

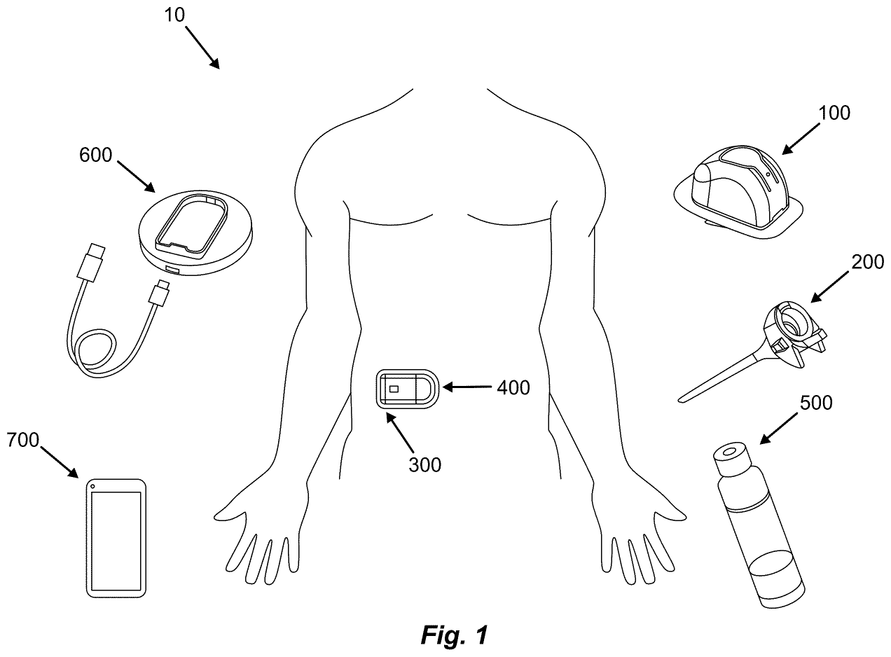

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula 200, pump 300, cap 400, cartridge 500, charging system 600 and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula 200 and a pad to a wearer and to deliver medication through cannula 200 via a patch pump coupled to the pad. Further details regarding such a system can be found in U.S. Patent Publication No. 2022/0379014, which is hereby incorporated herein by reference in its entirety.

Applicator 100 is configured to apply an adhesive pad to the wearer and, upon actuation, to insert cannula 200 into the wearer. The pad is configured to be secured to the wearer for a period of time, e.g., at least 3 days, 7-10 days, etc., and then may be replaced by a similar pad using a similar applicator. The pad may include a pad skeleton having one or more locking mechanisms that are configured to couple the pad to applicator 100 for insertion of cannula 200 or to the assembled pump for delivery of medication. Applicator 100 may include an internal component configured to support an insertion mechanism designed to insert cannula 200 through the skin of the wearer via rotational movement and to guide and orient cannula 200 during insertion.

Preferably, applicator 100 is designed to suppress noise during insertion. The insertion mechanism may include an applicator needle configured to pierce the wearer's skin and a biasing member, which may be coupled to one or more links configured to interact with cannula 200 and the applicator needle. Upon actuation by the wearer, the insertion mechanism preferably rotates and applies a distal force on cannula 200 and the applicator needle within cannula 200, such that cannula 200 is inserted through the wearer's skin. Cannula 200 may include a proximal cannula head configured to couple to one or more locking mechanisms on the pad skeleton and, at the same time, uncouple applicator 100 from the pad skeleton. The insertion mechanism further may be configured to continue rotating to withdraw the applicator needle from cannula 200 and to store the applicator needle within the applicator after cannula 200 is inserted.

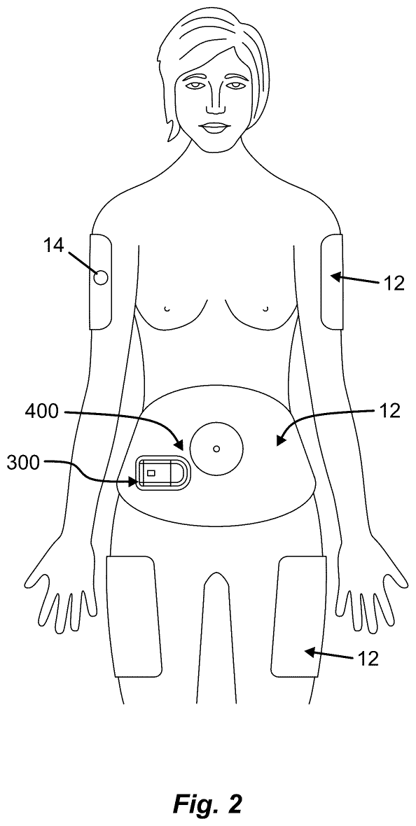

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

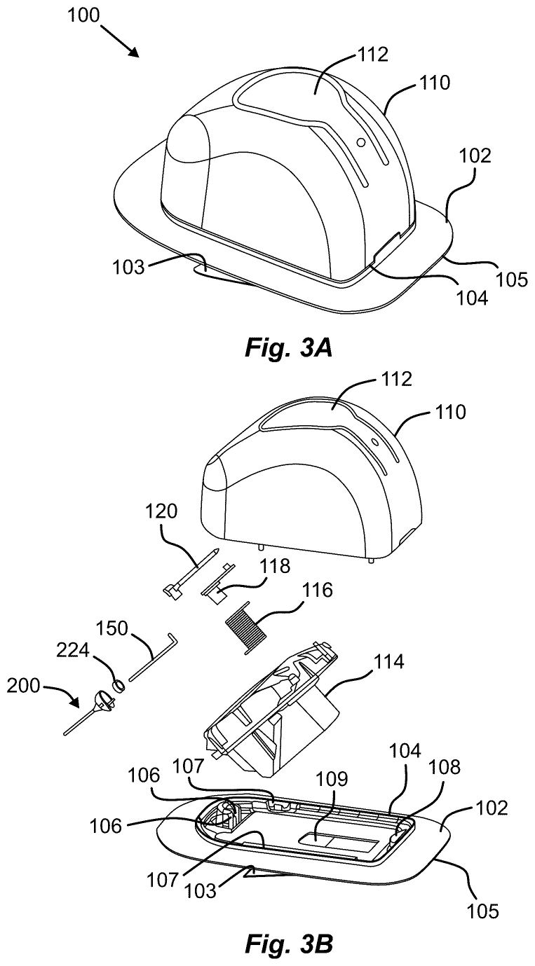

Referring now to FIGS. 3A and 3B, perspective and exploded views of an exemplary pad and applicator are described. Applicator 100 may transcutaneously apply a cannula, upon actuation by a user, which is designed to deliver doses of medication (e.g., insulin) from a patch pump configured to be removable coupled to the cannula. Advantageously, applicator further may apply a pad that is adhered to the wearer's skin and then coupled to the patch pump. For example, actuation of applicator 100 may both insert the cannula and cause the cannula to be locked to the adhesive pad in a single actuation. Further, applicator 100 may include internal components designed to minimize noise during the actuation process. For example, applicator 100 may avoid clicks and/or hard stops that make audible noises during insertion of the cannula.

In a pre-actuation state, applicator 100 may be coupled to pad 102 as shown in FIG. 3A. For example, applicator 100 may be coupled to pad 102 via pad skeleton 104 of pad, which is disposed on a first surface of pad 102. Skin-safe pad adhesive 105 may be disposed on a second, skin-facing surface of pad 102 such that the pump-pad assembly may be attached to a wearer for a period of time, for example, 3-5 days, 3-10 days, or 10 days or more. One or more release liners 103 may be attached to pad adhesive 105 until pad 102 is ready to be secured to the wearer. Pad skeleton 104 may be a frame with a shape designed to surround the pump-cap assembly so as to securely couple the adhesive pad to the pump-cap for wearing by the patient. Pad skeleton 104 may be designed to removably couple portions of pad 102 to applicator 100 in the pre-actuation state. For example, pad skeleton 104 may have one or more attachment mechanisms to lock pad 102 to applicator 100 and unlock upon actuation of applicator 100. Advantageously, the attachment mechanisms also may lock the cannula to pad 102 after actuation. As depicted in FIG. 3A, pad skeleton 104 may have pad attachments 106 at a first end of pad 102 and pad back clip 108 at a second end of pad 102. Pad attachments 106 and pad back clip 108 may interact with applicator 100 or a patch pump to lock the pad to applicator 100 or the patch pump. Pad attachments 106 may include at least two arms that protrude upwards from the pad and away from the skin surface of the wearer. Each arm may have an opening (e.g., slot) to receive extensions from the applicator during pre-actuation and extensions from the cannula post-actuation. Thus, the arms, which may have a U-shape, and openings may be used to lock to both the applicator and the cannula. Pad skeleton 104 may also include pad clips holes 107 disposed on the sides of pad skeleton 104. Pad clips holes 107 may be a hole or receptacle sized and shaped to interact with a corresponding feature of the pump-cap assembly such that the pump-cap assembly may be locked to the pad. Further, pad 102 may include pad opening 109 to allow direct sensing of the wearer's skin by one or more sensors of the pump. For example, the skin sensor(s) and/or the PPG sensor(s) may be positioned at pad opening 109 when the pump is coupled to the pad.

Applicator 100 may include applicator housing 110 and actuator 112. Applicator housing 110 is configured to house the mechanisms for inserting the cannula. After insertion of the cannula, internal component 114 is designed to withdraw and safely store the needle used to pierce the wearer's skin. Actuator 112, upon actuation, causes the cannula to be transcutaneously inserted into the wearer's skin. Actuation of actuator 112 also may unlock applicator 100 from pad 102. Actuation of actuator 112 also may lock the transcutaneously inserted cannula into pad 102. For example, actuation of applicator 100 may insert the cannula transcutaneously, unlock the applicator from the pad, and lock the cannula to the pad in a single actuation. Actuator 112 may release the internal mechanism disposed within applicator housing 110 when actuated by the wearer, thus causing the cannula to advance through the wearer's skin. Actuator 112 may be a button configured to be pressed by the wearer as illustrated, or may be a lever, snap, knob, or the like. The mechanism for inserting the cannula may include internal component 114, biasing member 1156, and links 118 and 120, which are disposed within applicator housing 110, and are configured to advance cannula 200 through pad 102 and into the wearer's skin. The mechanism may further include applicator needle 150, which is configured to be disposed within cannula 200 during insertion and withdrawn from cannula 200 after insertion. Self-sealing septum 224 may be disposed within the cannula head of cannula 200 in order to support and guide applicator needle 150 and minimize backflow out of cannula 200.

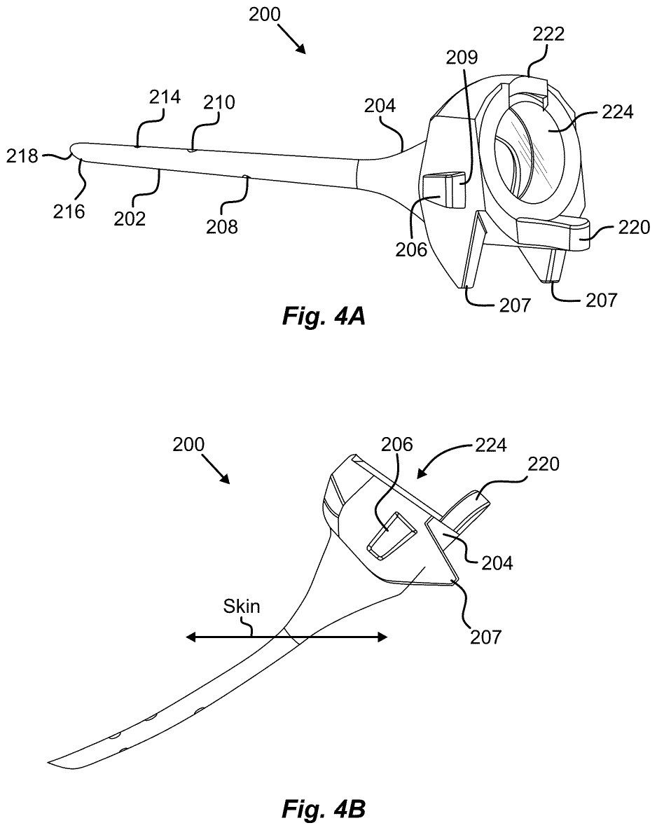

Referring now to FIGS. 4A and 4B, an exemplary cannula for delivering medication is described. Cannula 200 may be injection molded from a single piece of material, which is preferable to extrusion in order to reduce the risk of kinking of cannula 200. Cannula 200 preferably is made from a material that is insulin compatible and flexible and includes cannula head 204, cannula tip 218, and elongated shaft 202. Elongated shaft 202 is designed to be straight prior to deployment (FIG. 4A) and to curve, responsive to curving of the needle disposed therein, when inserted in the skin (FIG. 4B). The cross-section of cannula 200 may have an oval section shape along the extended shaft for better curve. In this manner, the cannula is thinner in the direction of the curve. Cannula head 204 is disposed at the proximal end of cannula 200 and configured to interact with the applicator needle and the needle through which the medication is delivered. Cannula tip 218 is disposed at the distal end of cannula 200 and may include distal aperture 216 for delivering medication. Elongated shaft 202 may extend between cannula head 204 and cannula tip 218 and may include one or more apertures 208, 210, 212, 214 for delivery of medication. Elongated shaft 202 may increase in diameter towards cannula head 204 and the wearer's skin surface, to mitigate the risk that the delivered medication travels proximally along the outside surface of the cannula to the dermal layer or the surface of the skin. This conical shape may also reduce the risk of kinking of cannula 200.

Cannula head 204 may include one or more applicator interfaces that are configured to interact with link 120 to permit rotational movement of the cannula during insertion of the cannula into the skin of the wearer. For example, applicator interface 220 may be disposed on the side of cannula head 204 that is farthest away from the skin surface of the wearer. Applicator interface 220 may be a rounded, convex protrusion, which interacts with a corresponding rounded, concave receptacle of link 120. Cannula head 204 also may include applicator interface 222, which may be disposed on the opposite side of the cannula head, the side closest to the skin surface of the wearer. Applicator interface 222 may be a rounded, concave receptacle, which interacts with a corresponding rounded, convex protrusion of link 120. The rounded shapes of applicator interfaces 220 and 222 and the corresponding features of link 120 are designed such that link 120 maintains smooth and continuous contact with cannula head 204 during insertion of cannula 200 into the wearer's skin.

Cannula head 204 may include one or more clips 206 configured to guide cannula 200 in a substantially linear direction. Clips may be any component of cannula head 204 that is configured to interact with the channel of the internal component of the applicator during insertion of cannula 200 into the wearer's skin. For example, clips 206 may be one or more wings disposed on a first and second side of cannula head 204 and sized and shaped to slide along the ledges of the channel. Clips 206 alternatively may be receptacles disposed on cannula head 204 and configured to slide along corresponding protrusions of the channel. Cannula head 204 may further include wings 207, which may be configured to interact with the guiding arm to order to prevent cannula 200 from rotating around the longitudinal axis of cannula 200 during and after insertion. Preferably, wings 207 are configured to protrude towards the wearer's skin and the guiding arm and are sized and shaped such that the guiding arm fits between the two wings. Clips 206 and wings 207 are designed to control orientation of the cannula during delivery and insertion. Because the apertures along the shaft of the cannula may be radially and longitudinally offset from one another, control of the orientation of the cannula in the wearer's skin is important to ensure precise delivery of medication through the aperture(s). Thus, clips 206 and wings 207 ensure axial orientation in a target direction of the apertures.

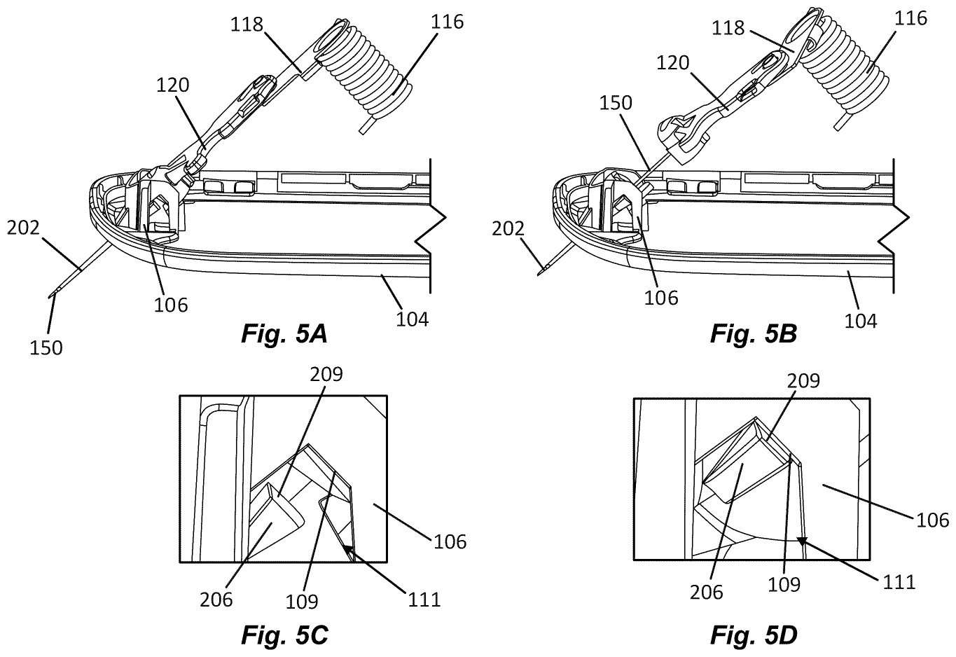

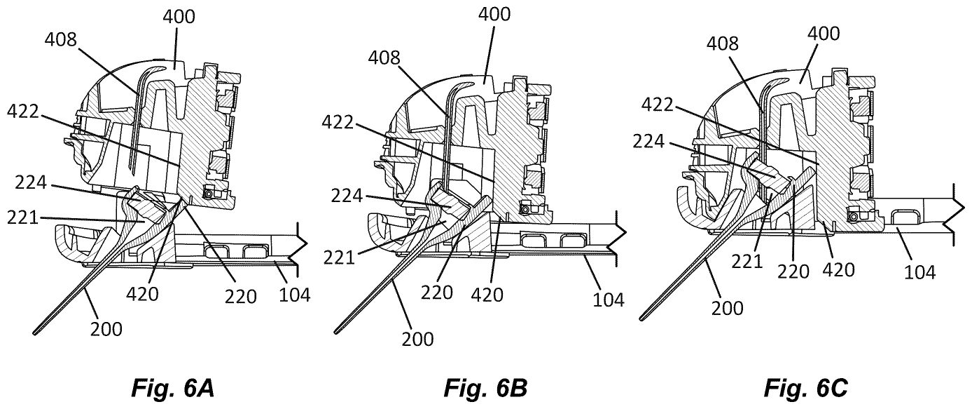

As noted above, upon actuation of applicator 100 the cannula is inserted into the user's skin and seated in the pad attachments 106 of pad skeleton 104. It can be a challenge to reliably and securely clip the cannula 200 into the pad skeleton 104 and to align the output needle 408 with the septum 224 in the cannula. FIGS. 5A-5B and 6A-6C depict further details of a system that reliably provides these functions.

Referring to FIGS. 5A-5D, a portion of an applicator 100 for a medication infusion system is schematically depicted illustrating features that cause the cannula 200 to reliably clip onto the pad skeleton 104. As noted above, in order to securely retain the cannula 200 on the pad skeleton, clips 206 on cannula must interlock within pad attachments 106 of pad skeleton 104. In particular, a rear surface 209 on the clip 206 on each side of cannula 200 (see also FIG. 4A) must be inserted through the aperture 111 in each pad attachment 106 and abut an interlocking surface 109 of pad attachment 106. In order to reliably ensure that this secure connection is made, the applicator 100 depicted in FIGS. 5A-5D has been designed to provide an increased depth of insertion that provides a greater separation between rear surface 209 of clip 206 and interlocking surface 109 of pad attachment 106. This provides a larger tolerance for interfacing the clips 206 with the pad attachments 106.

In particular, referring to FIGS. 5A and 5C, which depict the insertion needle 150 at a maximum insertion depth with link 118 having rotated with respect to biasing member 116 and link 120 fully extended from link 118, it can be seen that rear surface 209 of clip 206 has been advanced such that there is a relatively large margin of space between rear surface 209 and interlocking surface 109 of pad attachment 106. FIGS. 5B and 5D depict the applicator with the insertion needle 150 retracted. When the insertion needle is retracted, the cannula 200 naturally with draws slightly from the maximum insertion depth due to skin repulsion of the cannula and friction from retraction of the insertion needle 150 through the cannula 200. As can be seen most clearly in FIG. 5D, this causes the rear surfaces 209 of clips 206 to interlock with the interlocking surfaces 109 of the pad attachments 106 to securely retain the cannula 200 on the pad skeleton 104. This design provides a larger tolerance for interlocking the components by enabling the clips 206 to be inserted a larger distance past the interlocking surfaces 109 of the pad attachments 206 and then retract back to interlock with those surfaces.

This larger tolerance is necessary because the clearance between rear surfaces 209 of clips 206 and interlocking surfaces 109 of the pad attachments 106 is dependent on the tolerances of a number of components, namely, link 118, link 120, cannula 200, pad skeleton 104 and internal component 114. In particular, the overall tolerance must be sufficient so that even if all of the link 118, line 120 and cannula 200 are at the shortest end of the respective tolerances and the pad skeleton 104 and internal component 114 are at the longest end of the respective tolerances that there is still sufficient clearance to enable the rear surfaces 209 of clips 206 to interlock with the interlocking surfaces 109 of the pad attachments 106. The natural retraction that occurs following insertion will then cause clips 206 a distance past the interlocking surfaces 109 to retract against those surfaces. In addition, due to this larger tolerance, if each of the tolerances is at the opposite end (i.e., link 118, line 120 and cannula at longest and pad skeleton 104 and internal component 114 at longest), the cannula head can come into conflict with the far end of pad attachments 106. The system is also designed to provide a secure interlock when the clips 206 retract in this circumstance. These features therefore provide a more reliable and secure manner of attaching the cannula 200 to the pad skeleton 104.

Although the above features provide an improved and more reliable connection between the cannula 200 and the pad skeleton 104, it has been found that the greater tolerance of the cannula 200 that enables this enhanced reliability can lead to an inconsistent positioning of the septum 224 of the cannula 200. In particular, when the pump 300 and cap 400 are attached to the pad skeleton 104, the outflow needle 408 pierces the septum 224 to provide fluid communication between the patch pump and the cannula 200. The outflow needle 408 must be precisely located such that it extends through the septum 224 and into cavity 221 (shown in FIGS. 6A-6C, which are described in more detail below) within cannula head 204 beneath the septum 224 that puts the outflow needle 408 in fluid communication with the cannula shaft 202 and therefore the body of the patient. Any inconsistent positioning of the septum 204 when the pump 300 and cap 400 are attached could cause the outflow needle 408 to fail to be inserted in the proper location, which could lead either to an inadequate piercing or leakage through the septum. Referring now to FIGS. 6A-6C a cap 400 design includes features to provide a more precise piercing of the septum 224 by the outflow needle 408.

At the beginning of this insertion process depicted in FIG. 6A, with the outflow needle 408 positioned above the septum 224 following insertion of the cannula 200, an angled ramp 420 in the cap 400 abuts the proximal end of the applicator interface 220 of the cannula 200. The angled ramp 420 provides a distal force on cannula 200, that pushes the cannula 200 further into the skin (this not an issue because the cannula had previously been inserted to the insertion depth described above before retracting to interlock in the pad attachments 106). As the outflow needle 408 is advanced towards the septum 224, the angled ramp 420 is guided off of the proximal end of applicator interface 220 and a vertical ramp 422 abuts the applicator interface 220 as shown in FIG. 6B. The removal of the force provided by the angled ramp 420 also causes the cannula 200 to securely clip in the pad attachments 106 as shown in FIG. 5D, which prevents any further movement of the cannula 200 and ensures proper lateral alignment between the outflow needle 408 and the septum 224. The vertical ramp 422 may be generally parallel to the outflow needle 408 and be positioned to provide proper longitudinal alignment for outflow needle 408 and is guided along the applicator interface 220 on the cannula 200 to maintain that alignment as the outflow needle 408 is lowered into the cannula 220 to pierce the septum 224 as shown in FIG. 6C. In this manner, precise positioning of the septum 224 for piercing by the outflow needle 408 is therefore maintained despite the greater variability of the positioning of the cannula 200 and septum 224. Due to these features, the septum is able to be maintained in a fixed, predetermined position relative to the pump for insertion of the outflow needle therethrough.

The combination of these features therefore provides enhanced reliability of the cannula 200 properly clipping into the pad attachments 106 on the pad skeleton 104 while maintaining precise positioning of the outflow needle 408 through the septum 224 in the cannula 200. This is accomplished by provided a larger tolerance for location of the cannula on insertion of the cannula 200 to provide a more reliable interlocking of the cannula 200 with the pad skeleton 224 and by providing a smaller tolerance for location of the cannula (and therefore the septum) during pump attachment by controlling a location of the cannula 200 as the outflow needle 408 is brought into contact with the septum with features in the pump cap 400.

In embodiments, a medication infusion system includes a pad skeleton having a pad attachment. An applicator can be configured to attach the pad skeleton onto a body of a user and to attach a cannula including a septum to the pad attachment. The applicator can cause a variable positioning of the cannula with respect to the pad attachment on insertion of the cannula to provide a secure attachment between the cannula and the pad attachment. A patch pump can include a reservoir configured to contain a medicament and be configured to be attached to the pad skeleton to cause an outflow needle in fluid communication with the reservoir to pierce the septum to cause the reservoir to be in fluid communication with the cannula. The patch pump can be configured to control the variable positioning of the cannula as the septum is pierced by the outflow needle.

In some embodiments, the pad attachment comprises a pair of opposing apertures and the cannula assembly comprises a pair of opposing clips, and the secure attachment between the cannula assembly and the pad attachment includes the clips interlocking within the apertures.

In some embodiments, a planar rear surface of each clip abuts an interlocking surface defined by a corresponding aperture to lock the clips within the apertures.

In some embodiments, the applicator is configured to insert the cannula assembly through the skin of the user upon actuation to an insertion depth such that the planar rear surface of each clip is inserted passed the corresponding interlocking surface and wherein a natural reaction of the skin of the user following actuation causes the planar rear surfaces to move in an opposite direction to interlock with the interlocking surfaces.

In some embodiments, the variable positioning of the cannula assembly is caused by providing manufacturing tolerances in the applicator sufficient to ensure that the planar rear surface of each clip is inserted passed the corresponding interlocking surface a sufficient distance.

In some embodiments, the patch pump is configured to control the variable positioning of the cannula assembly by causing the cannula assembly to be in a predetermined, fixed position relative to the outflow needle.

In some embodiments, the patch pump is configured to cause the cannula assembly to be in a predetermined, fixed position relative to the outflow needle by engaging one or more surfaces of the patch pump with the cannula assembly to maintain the cannula assembly in the predetermined, fixed position.

In some embodiments, the one or more surfaces include an angled ramp surface on the patch pump engaging with a proximal applicator interface on the cannula assembly to provide a distal force on the cannula assembly that pushes the cannula assembly further into the skin of the user.

In some embodiments, the one or more surfaces include a vertical ramp surface on the patch pump engaging with a proximal applicator interface, the vertical ramp surface maintaining longitudinal alignment of the outflow needle when the outflow needle is engaged with the septum.

In some embodiments, the vertical ramp surface is generally parallel with the outflow needle.

In embodiments, a medication infusion system can include a pad skeleton having a pad attachment and an applicator configured to attach the pad skeleton onto a body of a user and to attach a cannula assembly to the pad attachment. The applicator can be configured to insert the cannula assembly into a skin of the user to an insertion depth such that a natural reaction of the skin following insertion causes the cannula assembly to move in a direction opposite of a direction of insertion to interlock the cannula assembly with the pad attachment.

In some embodiments, the interlock is provided by a pair of opposing clips on the cannula assembly interlocking within a pair of opposing apertures in the pad attachment.

In some embodiments, a planar rear surface of each clip abuts an interlocking surface defined by a corresponding aperture to interlock the clips within the apertures.

In some embodiments, at the insertion depth the planar rear surface of each clip is inserted passed the corresponding interlocking surface and the natural reaction of the skin of the user following actuation causes the planar rear surfaces to move in the opposite direction to interlock with the interlocking surfaces.

In embodiments, a medication infusion system can include a pad skeleton having a pad attachment configured to receive a cannula assembly having a septum therein, the cannula assembly having a variable positioning with respect to the pad attachment. A patch pump including a reservoir configured to contain a medicament can be configured to be attached to the pad skeleton to cause an outflow needle in fluid communication with the reservoir to pierce the septum to cause the reservoir to be in fluid communication with the cannula assembly, wherein the patch pump is configured to control the variable positioning of the cannula assembly as the septum is pierced by the outflow needle.

In some embodiments, the patch pump is configured to control the variable positioning of the cannula assembly by causing the cannula assembly to be in a predetermined, fixed position relative to the outflow needle.

In some embodiments, the patch pump is configured to cause the cannula assembly to be in a predetermined, fixed position relative to the outflow needle by engaging one or more surfaces of the patch pump with the cannula assembly to maintain the cannula assembly in the predetermined, fixed position.

In some embodiments, the one or more surfaces include an angled ramp surface on the patch pump engaging with a proximal applicator interface on the cannula assembly to provide a distal force on the cannula assembly that pushes the cannula assembly further into the skin of the user.

In some embodiments, the one or more surfaces include a vertical ramp surface on the patch pump engaging with a proximal applicator interface on the cannula assembly, the vertical ramp surface maintaining longitudinal alignment of the outflow needle when the outflow needle is engaged with the septum.

In some embodiments, the vertical ramp surface is generally parallel with the outflow needle.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. A medication infusion system, comprising:
a pad skeleton having a pad attachment;
an applicator configured to attach the pad skeleton onto a body of a user and to attach a cannula assembly including a septum to the pad attachment, wherein the applicator causes a variable positioning of the cannula assembly with respect to the pad attachment on insertion of the cannula assembly to provide a secure attachment between the cannula assembly and the pad attachment;
a patch pump including a reservoir configured to contain a medicament, the patch pump configured to be attached to the pad skeleton to cause an outflow needle in fluid communication with the reservoir to pierce the septum to cause the reservoir to be in fluid communication with the cannula assembly, wherein the patch pump is configured to control the variable positioning of the cannula assembly as the septum is pierced by the outflow needle.

2. The medication infusion system of claim 1, wherein the pad attachment comprises a pair of opposing apertures and the cannula assembly comprises a pair of opposing clips, and wherein the secure attachment between the cannula assembly and the pad attachment includes the clips interlocking within the apertures.

3. The medication infusion system of claim 2, wherein a planar rear surface of each clip abuts an interlocking surface defined by a corresponding aperture to lock the clips within the apertures.

4. The medication infusion system of claim 3, wherein the applicator is configured to insert the cannula assembly through the skin of the user upon actuation to an insertion depth such that the planar rear surface of each clip is inserted passed the corresponding interlocking surface and wherein a natural reaction of the skin of the user following actuation causes the planar rear surfaces to move in an opposite direction to interlock with the interlocking surfaces.

5. The medication infusion system of claim 3, wherein the variable positioning of the cannula assembly is caused by providing manufacturing tolerances in the applicator sufficient to ensure that the planar rear surface of each clip is inserted passed the corresponding interlocking surface a sufficient distance.

6. The medication infusion system of claim 1, wherein the patch pump is configured to control the variable positioning of the cannula assembly by causing the cannula assembly to be in a predetermined, fixed position relative to the outflow needle.

7. The medication infusion system of claim 1, wherein the patch pump is configured to cause the cannula assembly to be in a predetermined, fixed position relative to the outflow needle by engaging one or more surfaces of the patch pump with the cannula assembly to maintain the cannula assembly in the predetermined, fixed position.

8. The medication infusion system of claim 7, wherein the one or more surfaces include an angled ramp surface on the patch pump engaging with a proximal applicator interface on the cannula assembly to provide a distal force on the cannula assembly that pushes the cannula assembly further into the skin of the user.

9. The medication infusion system of claim 7, wherein the one or more surfaces include a vertical ramp surface on the patch pump engaging with a proximal applicator interface, the vertical ramp surface maintaining longitudinal alignment of the outflow needle when the outflow needle is engaged with the septum.

10. The medication infusion system of claim 9, wherein the vertical ramp surface is generally parallel with the outflow needle.

11. A medication infusion system, comprising:
a pad skeleton having a pad attachment; and
an applicator configured to attach the pad skeleton onto a body of a user and to attach a cannula assembly to the pad attachment, wherein the applicator is configured to insert the cannula assembly into a skin of the user to an insertion depth such that a natural reaction of the skin following insertion causes the cannula assembly to move in a direction opposite of a direction of insertion to interlock the cannula assembly with the pad attachment.

12. The medication infusion system of claim 11, wherein the interlock is provided by a pair of opposing clips on the cannula assembly interlocking within a pair of opposing apertures in the pad attachment.

13. The medication infusion system of claim 12, where a planar rear surface of each clip abuts an interlocking surface defined by a corresponding aperture to interlock the clips within the apertures.

14. The medication infusion system of claim 13, wherein at the insertion depth the planar rear surface of each clip is inserted passed the corresponding interlocking surface and the natural reaction of the skin of the user following actuation causes the planar rear surfaces to move in the opposite direction to interlock with the interlocking surfaces.

15. A medication infusion system, comprising:
a pad skeleton having a pad attachment configured to receive a cannula assembly having a septum therein, the cannula assembly having a variable positioning with respect to the pad attachment;
a patch pump including a reservoir configured to contain a medicament, the patch pump configured to be attached to the pad skeleton to cause an outflow needle in fluid communication with the reservoir to pierce the septum to cause the reservoir to be in fluid communication with the cannula assembly, wherein the patch pump is configured to control the variable positioning of the cannula assembly as the septum is pierced by the outflow needle.

16. The medication infusion system of claim 15, wherein the patch pump is configured to control the variable positioning of the cannula assembly by causing the cannula assembly to be in a predetermined, fixed position relative to the outflow needle.

17. The medication infusion system of claim 16, wherein the patch pump is configured to cause the cannula assembly to be in a predetermined, fixed position relative to the outflow needle by engaging one or more surfaces of the patch pump with the cannula assembly to maintain the cannula assembly in the predetermined, fixed position.

18. The medication infusion system of claim 17, wherein the one or more surfaces include an angled ramp surface on the patch pump engaging with a proximal applicator interface on the cannula assembly to provide a distal force on the cannula assembly that pushes the cannula assembly further into the skin of the user.

19. The medication infusion system of claim 17, wherein the one or more surfaces include a vertical ramp surface on the patch pump engaging with a proximal applicator interface on the cannula assembly, the vertical ramp surface maintaining longitudinal alignment of the outflow needle when the outflow needle is engaged with the septum.

20. The medication infusion system of claim 19, wherein the vertical ramp surface is generally parallel with the outflow needle.
